(19) United States
(12) Patent Application Publication
(10) Pub. No.: US 2019/0380924 A1
(43) Pub. Date: Dec. 19, 2019

(54) COSMETIC FOAM MADE FROM AN EMULSION CONTAINING GLYCERINE AND ALCOHOL

(71) Applicant: BEIERSDORF AG, Hamburg (DE)

(72) Inventors: Kerstin SKUBSCH, Prisdorf (DE); Claudia MUELLER, Tangstedt (DE); Sabine SCHULZ, Hamburg (DE); Julia ECKERT, Hamburg (DE); Katrin RUPP, Hamburg (DE)

(21) Appl. No.: 15/772,845

(22) PCT Filed: Oct. 17, 2016
(86) PCT No.: PCT/EP2016/074834

§ 371 Date: Sep. 24, 2018

(30) Foreign Application Priority Data
Nov. 4, 2015 (DE) 102015221568.7

(51) Int. Cl. A61K 8/04; A61K 8/06; A61K 8/34; A61K 8/31; A61K 8/92; A61K 8/35; A61K 8/44; A61K 8/37; A61K 8/362; A61K 8/67; A61K 8/20; A61Q 19/10

(52) U.S. Cl. CPC A61K 8/046; A61Q 19/10; A61K 8/345; A61K 8/31; A61K 8/922; A61K 8/92; A61K 8/355; A61K 8/44; A61K 8/37; A61K 8/362; A61K 8/342; A61K 8/34; A61K 8/347; A61K 8/678; A61K 8/20; A61K 8/062

(57) ABSTRACT

The invention relates to a cosmetic foam made from a) an O/W emulsion containing glycerine and b) mono- and/or polyvalent alcohols with a carbon chain length of less than 12 C-atoms and c) a gas or gas mixture of propane, n-butane and/or isobutane, foaming the emulsion, which emulsion is fre of polyethylene glycol derivatives (PEG derivatives).

The present invention relates to a cosmetic foam of an O/W emulsion comprising glycerol and mono- and/or polyhydric alcohols having a carbon chain length of less than 12 C atoms and a gas or gas mixture of propane, n-butane and/or isobutane which foams the emulsion, wherein the emulsion is free of polyethylene glycol derivatives (PEG derivatives). The invention further relates to an aerosol can with release valve containing such a foam.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. Active ingredients are commonly added thereto, which are intended to regenerate the skin and for example to prevent and reduce the premature aging thereof (e.g. the appearance of fine lines and wrinkles).

Skin care products (also some skin cleansing products) generally consist of emulsions. Emulsions are generally understood to mean heterogeneous systems composed of two liquids that are immiscible, or of only limited miscibility, with each other, which are typically referred to as phases and in which one of the two liquids is dispersed in the other liquid in the form of fine droplets. Externally and viewed with the naked eye, emulsions appear homogeneous.

If the two liquids are water and oil and oil droplets are present finely distributed in water, it is an oil-in-water emulsion (O/W emulsion, milk for example). The basic character of an O/W emulsion is influenced by the water. The reverse principle applies to a water-in-oil emulsion (W/O emulsion, butter for example), wherein the basic character in this case is determined by the oil.

In addition to the “classical” application, where emulsions are applied to the skin directly from the storage container, there are also a smaller number of applications in which the emulsion is foamed with the aid of a propellant for application to the skin. Foamed emulsions, by virtue of their foam consistency, have a particular skin feel that is perceived by the users as better distributable on the skin with a lighter and less sticky skin feel. The application of the mousse texture is also fun for the users.

The disadvantage of the prior art, however, is the fact that polyethylene glycols and/or polyethylene glycol derivatives have to be added to the preparations for producing cosmetic foam emulsions. PEG derivatives are compounds having alcohol or acid functions which are fully or partially etherified or esterified in order to keep the foam stable over a longer period. However, these PEG derivatives are increasingly undesirable to consumers since some scientists question their harmlessness to health. Whether these concerns have a scientific basis remains an open question in the context of the present invention. The reality, however, is that consumers increasingly demand cosmetics which are “PEG-free”.

It was therefore an object of the invention to develop a sensorially attractive cosmetic foam, based on a foamed emulsion, that is stable over a relatively long period, which is “PEG-free”.

Cosmetic O/W emulsions also have the disadvantage that they have to be particularly strongly preserved in order for them to be microbiologically safe over a relatively long period of time. For this purpose, preservatives, particularly from the group of parabens, are used. Preservatives and particularly parabens (whether correctly or incorrectly remains an open question in the context of this disclosure) have a reputation among consumers of not being entirely harmless with regard to their compatibility in terms of health and the environment.

It was therefore an object of the invention to develop a microbiologically stable cosmetic foam, based on an O/W emulsion, which is “paraben-free”.

It is also known per se to those skilled in the art that it is possible to use alcohols to preserve cosmetic O/W emulsions, for example ethanol or alkanediols such as 1,2-octanediol (caprylyl glycol). However, it is known that alcohols destabilize the foam of a foamed emulsion such that this rapidly collapses. Moreover, ethanol in particular is irritating to the skin and has a tendency to desiccate the skin.

It was therefore an object of the present invention to develop a microbiologically stable “paraben-free” foam based on an O/W emulsion which in any case has a low potential as a skin irritant and results in a stable foam.

Finally, foams foamed with propane, n-butane (“butane”) or isobutane are generally relatively highly inflammable. This problem is enhanced if the preparation comprises further flammable ingredients such as ethanol. For such systems according to the prior art, particular fire-protection measures have to be taken in the preparation, storage, storage in the storage container and during application.

It was therefore the object of the present invention to develop an O/W emulsion foamed or foamable with propane, n-butane (“butane”) or isobutane in which no particular fire-protection measures have to be observed.

The object is achieved, surprisingly, by a cosmetic foam comprising

a) an O/W emulsion comprising glycerol and

b) mono- and/or polyhydric alcohols having a carbon chain length of less than 12 C atoms and

c) a gas or gas mixture of propane, n-butane and/or isobutane which foams the emulsion,

wherein the emulsion is free of polyethylene glycol derivatives (PEG derivatives).

In this case, glycerol, propylene glycol and butylene glycol do not count as the mono- and/or polyhydric alcohols having a carbon atom chain length of less than 12 carbon atoms in accordance with the invention (definition).

Although EP1277455, EP1014916 and DE10138495 are known to those skilled in the art, these documents could not have indicated the approach of the present invention.

It is of particular advantage in accordance with the invention if the emulsion is free of soaps (an emulsion in accordance with the invention is “free of soaps” if it comprises less than 0.2% by weight of soaps, based on the total weight of the emulsion). In accordance with the invention, “soaps” are understood to mean sodium or potassium salts of fatty acids.

It is likewise of particular advantage in accordance with the invention if the emulsion is free of surfactants having an HLB value of greater than 12, wherein sodium cetearyl sulfate is not counted among the surfactants in accordance with the invention.

This fact is all the more surprising since soaps and surfactants are typically required for stabilizing cosmetic foams. In contrast, omitting the surfactants in the foams according to the invention leads to a stabilization of the foam and to a distinctly creamier foam which is more easily distributed.

In this advantageous embodiment according to the invention, the foams according to the invention are surprisingly more stable and creamier than when these surface-active compounds are added and have a significantly lower potential for skin irritation.

It is of advantage in accordance with the invention if the cosmetic is characterized in that the emulsion comprises from 3 to 20% by weight glycerol, based on the total weight of the emulsion. In this case, preferred in accordance with the invention is a content from 5 to 15% by weight, based on the total weight of the emulsion.

In addition, it is advantageous in accordance with the invention in the foam according to the invention if the total concentration of mono- and polyhydric alcohols having a carbon chain length of less than 12 carbon atoms in the emulsion is 0.5 to 15% by weight, based on the total weight of the emulsion. This total concentration corresponds to the single concentration in the case that the emulsion comprises only one alcohol having a carbon chain length of less than 12 carbon atoms.

Advantageous foams according to the invention are characterized in that the mono- and/or polyhydric alcohols having a carbon chain length of less than 12 carbon atoms are selected from the group of the compounds ethanol, phenoxyethanol, 1,2-pentanediol, 1,2-hexanediol, 1,2-octanediol, 2-methyl-1,3-propanediol, 1,2-decanediol, ethylhexylglycerol, benzyl alcohol, phenethyl alcohol.

If the foam according to the invention comprises ethanol, it is preferable in accordance with the invention to use this substance at a concentration of from 0.5 to 10% by weight, based on the total weight of the emulsion.

If the foam according to the invention comprises phenoxyethanol, it is preferable in accordance with the invention to use this substance at a concentration of from 0.1 to 1% by weight, based on the total weight of the emulsion.

If the foam according to the invention comprises 1,2-pentanediol, it is preferable in accordance with the invention to use this substance at a concentration of from 0.1 to 5% by weight, based on the total weight of the emulsion.

If the foam according to the invention comprises 1,2-hexanediol, it is preferable in accordance with the invention to use this substance at a concentration of from 0.1 to 5% by weight, based on the total weight of the emulsion.

If the foam according to the invention comprises 1,2-octanediol, it is preferable in accordance with the invention to use this substance at a concentration of from 0.1 to 5% by weight, based on the total weight of the emulsion.

If the foam according to the invention comprises 2-methyl-1,3-propanediol, it is preferable in accordance with the invention to use this substance at a concentration of from 0.5 to 10% by weight, based on the total weight of the emulsion.

If the foam according to the invention comprises 1,2-decanediol, it is preferable in accordance with the invention to use this substance at a concentration of from 0.1 to 5% by weight, based on the total weight of the emulsion.

If the foam according to the invention comprises ethylhexylglycerol, it is preferable in accordance with the invention to use this substance at a concentration of from 0.1 to 2% by weight, based on the total weight of the emulsion.

If the foam according to the invention comprises benzyl alcohol, it is preferable in accordance with the invention to use this substance at a concentration of from 0.1 to 2% by weight, based on the total weight of the emulsion.

If the foam according to the invention comprises phenethyl alcohol, it is preferable in accordance with the invention to use this substance at a concentration of from 0.1 to 3% by weight, based on the total weight of the emulsion.

Embodiments of the present invention that are advantageous according to the invention are characterized in that the foam is formed from 90-96% by weight emulsion and 4 to 10% by weight gas or gas mixture. Preferred embodiments in accordance with the invention are characterized in that the foam is formed from 92-94% by weight emulsion and 6 to 8% by weight gas or gas mixture. That the foams according to the invention can be uniformly foamed even with such low amounts of gas was not foreseeable to those skilled in the art. The reason for this is the surprisingly good and simple uniform distribution of the gas in the emulsion.

It is advantageous in accordance with the invention if the gas used is a gas mixture of butane, isobutane and/or propane. The mixing ratio of the gases is varied depending on the pressure rating, e.g.:

pressure rating 2.7 bar: 60% butane, 20% propane and 20% isobutane.

Pressure rating 3.0 bar: 5.3% butane, 15.3% propane and 79.4% isobutane.

Pressure rating 3.5 bar: 5% butane, 23% propane and 72% isobutane.

Preferred in accordance with the invention are pressure ratings of 2.7 bar, 3.0 bar and 3.5 bar.

Particular preference is given to pressure ratings of 3.0 bar and 3.5 bar.

It is of advantage according to the invention if the emulsion is free of parabens, isothiazolinones and 3-iodopropargyl N-butylcarbamate (I PBC) and also halogen-containing compounds.

Embodiments of the present invention that are advantageous according to the invention are also obtained when the emulsion comprises EDTA and/or betaine.

If the emulsion contains EDTA, it is advantageous according to the invention when the content of EDTA is from 0.01 to 2% by weight, based on the total weight of the emulsion.

If the emulsion contains betaine, it is advantageous according to the invention when the content of betaine is from 0.001 to 6% by weight, based on the total weight of the emulsion.

Embodiments of the present invention that are advantageous according to the invention are characterized in that the lipid phase of the emulsion comprises one or more fatty alcohols, almond oil, cocoa butter and/or shea butter. It has been particularly surprising in this case that the emulsions, when they comprise stearyl alcohol or behenyl alcohol, result in preparations that are readily and uniformly miscible with the gas, since both fatty alcohols lead to an increase in the viscosity of the emulsion which in principle should impede the miscibilty with the gas (gas mixture).

The concentration used advantageously in accordance with the invention of stearyl alcohol is from 0.01 to 4% by weight, based on the total weight of the emulsion.

The concentration used advantageously in accordance with the invention of behenyl alcohol is from 0.01 to 4% by weight, based on the total weight of the emulsion.

The concentration used advantageously in accordance with the invention of almond oil is from 0.01 to 5% by weight, based on the total weight of the emulsion.

The concentration used advantageously in accordance with the invention of cocoa butter is from 0.01 to 5% by weight, based on the total weight of the emulsion.

The concentration used advantageously in accordance with the invention of shea butter is from 0.01 to 5% by weight, based on the total weight of the emulsion.

Embodiments of the present invention that are advantageous according to the invention are characterized in that the proportion by weight of the lipid phase of the emulsion is 7 to 25% by weight, based on the total weight of the emulsion.

It is of advantage in accordance with the invention if the emulsion comprises neither mineral oil nor silicone oil.

Silicone oils may be present however also in amounts of less than 1% by weight, based on the total weight of the emulsion.

It is advantageous in the context of the present invention if the cosmetic foam is characterized in that the emulsion is free of thickeners such as silicon dioxide, aluminum silicates, polysaccharides or derivatives thereof, e.g. hyaluronic acid, xanthan gum, hydroxypropyl methylcellulose, polyacrylates such as carbopols, for example carbomers and acrylates/C10-30 alkyl acrylate crosspolymer.

The water phase of the emulsion according to the invention may comprise customary auxiliaries.

Embodiments of the present invention that are advantageous according to the invention are characterized in that the emulsion comprises one or more emulsifiers selected from the group of the compounds polyglycerol esters such as polyglyceryl-10 stearate or polyglyceryl-3 methylglucose distearate, cetearyl glycoside, sodium stearoyl glutamate, glyceryl stearate citrate, potassium cetyl phosphate, stearic acid salts or a combination of sodium cetearyl sulfate and glyceryl stearate SE.

It is of advantage in accordance with the invention if the emulsion according to the invention comprises emulsifiers at a total concentration of 0.1 to 8% by weight, based on the total weight of the emulsion. This concentration corresponds to the advantageous single concentration in the case that the emulsion comprises only one emulsifier.

If the emulsion according to the invention comprises glyceryl stearate citrate as emulsifier, it is preferable in accordance with the invention to use this emulsifier at a concentration of 0.5 to 5% by weight, based on the total weight of the emulsion.

If the emulsion according to the invention comprises cetyl phosphate as emulsifier, it is preferable in accordance with the invention to use this emulsifier at a concentration of 0.05 to 2% by weight, based on the total weight of the emulsion.

If the emulsion according to the invention comprises stearic acid salts as emulsifier, it is preferable in accordance with the invention to use this emulsifier at a concentration of 0.05 to 0.5% by weight, based on the total weight of the emulsion.

In this case, preference is given to using sodium and potassium salts as stearic acid salts according to the invention.

If the emulsion comprises a combination of sodium cetearyl sulfate and glyceryl stearate SE as emulsifier, it is preferable in accordance with the invention to use these substances at the following concentrations:

It is of advantage in accordance with the invention if the emulsion according to the invention comprises sodium cetearyl sulfate in an amount of 0.1 to 2% by weight, based on the total weight of the emulsion.

It is of advantage in accordance with the invention if the emulsion according to the invention comprises glyceryl monostearate SE in an amount of 0.1 to 5% by weight, based on the total weight of the emulsion.

Self-emulsifying glyceryl monosterate with the INCI Glyceryl Monostearate SE is used in accordance with the invention.

Advantageously in accordance with the invention, the ratio by weight in the emulsion of sodium cetearyl sulfate to glyceryl monostearate is from 1:1 to 1:20.

The emulsion according to the invention can also advantageously comprise salts, especially sea salt.

It is advantageous in accordance with the invention if the emulsion according to the invention comprises one or more active ingredients selected from the group of compounds comprising gylcyrrhetic acid, urea, arctiin, alpha-lipoic acid, folic acid, phytoene, D-biotin, coenzyme Q10, hyaluronic acid, alpha-glucosylrutin, carnitine, carnosine, caffeine, natural and/or synthetic isoflavonoids, glycerylglucose, creatine, creatinine, taurine, β-alanine and/or licochalcone A, panthenol, tocopherol, tocopherol acetate, vitamin C, vitamin C derivatives, Glycyrrhiza inflata root extract, magnolia extract.

Moreover, embodiments of the present invention that are advantageous according to the invention are characterized in that the preparation contains propylene glycol, butylene glycol, 2-methylpropan-1,3-diol, ethylhexylglycerol, 1,2-pentanediol, 1,2-hexanediol, 1,2-octanediol and/or 1,2-decanediol. In this case, the content of 2-methylpropan-1,3-diol, 1,2-pentanediol and/or 1,2-hexanediol is preferred according to the invention.

Embodiments of the present invention that are advantageous according to the invention are characterized in that the emulsion comprises one or more UV filters selected from the group of the compounds 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene)camphor; 3-benzylidenecamphor; ethylhexyl salicylate; terephthalidene dicamphor sulfonic acid; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; 4-(tert-butyl)-4′-methoxydibenzoylmethane; homomenthyl salicylate; 2-ethylhexyl 2-hydroxybenzoate; dimethicodiethylbenzalmalonate; 3-(4-(2,2-bis ethoxycarbonylvinyl)phenoxy)propenyl)methoxysiloxane/dimethylsiloxane—copolymer; 4-(tert-butyl)-4′-methoxydibenzoylmethane; hexyl 2-(4′-diethylamino-2′-hydroxybenzoyl)benzoate; dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

It is particularly preferable in accordance with the invention if the emulsion is free of propylparaben and butylparaben, 3-iodo-2-propynyl butylcarbamate, 3-(4-methylbenzylidene)camphor, 2-hydroxy-4-methoxybenzophenone (oxybenzone) and halogen-containing compounds.

The cosmetic emulsions according to the invention may also advantageously comprise fillers, although not mandatory, which further improve, for example, the sensory and cosmetic properties of the formulations and which generate or enhance, for example, a velvety or silky skin feel. Advantageous fillers in the context of the present invention are starch and starch derivatives (such as tapioca starch, distarch phosphate, aluminum or sodium starch octenyl succinate and the like), pigments, which have principally neither UV filter nor staining effects, and/or Aerosils® (CAS No. 7631-86-9) and/or talc, polymethylsilsesquioxane, nylon, silica dimethyl silylate.

It is preferable in accordance with the invention if the cosmetic foam is characterized in that the emulsion comprises tapioca starch modified with polymethylsilsequioxane (INCI Tapioca starch+Polymethylsilsesquioxane). This is advantageous in accordance with the invention at a concentration of 0.1 to 3% by weight, based on the total weight of the emulsion.

The foam according to the invention or the emulsion according to the invention advantageously have in accordance with the invention a pH of 5 to 7.5.

Advantageously in accordance with the invention, the foam according to the invention is stored in an aerosol can with release valve and is applied therefrom. For application, the mixture of emulsion and gas(es) according to the invention is firstly mixed thoroughly by shaking in the aerosol can and then withdrawn via the release valve and applied to the skin. In this case, a further advantage in accordance with the invention is shown in that the foam according to the invention can be more completely withdrawn from the aerosol can in comparison with conventional foams, i.e. the so-called “residual emptying” is surprisingly higher than comparable conventional foams. Moreover, the foam according to the invention exhibits a surprisingly good packaging compatibility even at low pH, i.e. corrosion effects, for example, occur less frequently and are weaker than comparable conventional systems.

It is possible to use customary known aerosol can systems as aerosol cans with release valves.

An aerosol can with release valve comprising the foam according to the invention is therefore also in accordance with the invention, and also a method for applying cosmetic foams to the skin, which is characterized in that the mixture of emulsion and gas(es) according to the invention is firstly mixed thoroughly by shaking in the aerosol can and is subsequently withdrawn via the release valve and applied to the skin.

Finally, it is preferable in accordance with the invention if the foam according to the invention is characterized in that it is contained in an aerosol can with release valve.

### Comparative Experiment

| Composition | Sample A | Sample B |
|---|---|---|
| Caprylic/Capric Triglyceride | 3 | 3 |
| C15-19 Alkane | 4 | 4 |
| Isopropyl Palmitate | 3 | 3 |
| Shea Butter | 1 | 1 |
| Dimethicone | 1 | 1 |
| Glyceryl Stearate SE | 1.5 | |
| Sodium Cetearyl Sulfate | 0.15 | |
| PEG-40 Stearate | | 1.65 |
| Glycerol | 9.3 | 9.3 |
| 45% aqueous sodium hydroxide | 0.05 | 0.05 |
| solution | | |
| Phenoxyethanol | 0.5 | 0.5 |
| Behenyl Alcohol | 2 | 2 |
| Carbomer | 0.1 | 0.1 |
| Water | 74.4 | 74.4 |
| Preservation stress test | | |
| | Microbial count after 14 d | |
| | CFU | |
| Staph.areus, Staph.epidermis | 71000 | 200000 |
| Inoculation 170000 CFU | | |
| Pseudomonas aeruginosa | 700 | 1900 |
| Pseudomonas putida | | |
| Burkholderia cepacia | | |
| Inoculation: 290000 | | |
| Escherichia coli | 6900 | >200000 |
| Enetrobacter gergoviae | | |
| Klebsiella pneumoniae | | |
| Inoculation: 410000 | | |
| Candida albicans | 160000 | 160000 |
| Candida parapsilosis | | |
| Pichia guilliermondii | | |
| Inoculation: 320000 | | |
| Aspergillus brasiliensis | 320000 | 350000 |
| Inoculation: 240000 | | |
| Stability: | | |
| Emulsion stored for 2 months at | OK | Distinct water |
| 40° C. | | separation |
| Foam | | |
| 96% Emulsion + 4% | Creamy, | Aqueous, |
| propane/butane/isobutane | caring fine- | mid-sized |
| Pressure rating 2.7 bar | bubble foam | bubble |
| | | unstable |
| | | foam |

### Comparative Experiment II

| Composition | Sample #40 | Sample #41 |
|---|---|---|
| Piroctone olamine | 0.06 | 0.06 |
| C15-19 Alkane | 4 | 4 |
| Shea Butter | 1 | 1 |
| Mineral oil | 3 | 3 |
| Dimethicone | 1 | 1 |
| Isopropyl Palmitate | 3 | 3 |
| Glyceryl Stearate SE | 1.5 | 1.5 |
| Sodium Cetearyl Sulfate | 0.15 | 0.15 |
| Perfume | 0.35 | 0.35 |
| Glycerol | 9.3 | 9.3 |
| 45% aqueous sodium hydroxide | 0.06 | 0.06 |
| solution | | |
| Phenoxyethanol | 0.9 | 0.9 |
| Behenyl Alcohol | 2 | 2 |
| Carbomer | 0.1 | 0.1 |
| Water | 73.58 | 72.58 |
| Ethanol | | 1 |
| Stability: | | |
| Emulsion stored for 6 months at | OK | OK |
| 40° C. | | |
| Foam | | |
| 94% Emulsion + 6% | Unstable, | Creamy, |
| propane/butane/isobutane | coarse- | stable fine- |
| Pressure rating 3.0 bar | bubble foam | bubble foam |

### EXAMPLES

The examples below are intended to illustrate the present invention without limiting it. Unless otherwise stated, all quantitative data, fractions and percentages are based on the weight and the total amount or on the total weight of the preparations.

| | C | D | E | F |
|---|---|---|---|---|
| Composition in % | m [%] | m [%] | m [%] | m [%] |
| Sodium Cetearyl Sulfate | 0.15 | 0.2 | 0.1 | 0.15 |
| Glyceryl Stearate SE | 1.5 | 1 | 2 | 1.5 |
| Isopropyl Stearate | 1 | | | 1 |
| Caprylic/Capric | | | | |
| Triglyceride | 3 | 6 | 3 | 3 |
| Octyldodecanol | 4 | | 3 | 4 |
| Mineral oil | | 3 | 3 | |
| Almond oil | 0.5 | | | |
| Shea Butter | 1 | 1 | 0.5 | 1 |
| Cocoa butter | | | 0.5 | |
| Tocopherol acetate | | | 0.2 | |
| Sea salt | | 0.1 | | |
| Behenyl alcohol | 2 | 2 | 2 | 2 |
| Perfume | 0.3 | 0.35 | 0.3 | 0.35 |
| Glycerol | 5 | 7 | 9 | 10 |
| Ethylhexylglycerol | 0.5 | | | |
| Phenoxyethanol | | 0.6 | 1.0 | |
| Ethanol | 1 | 2 | 3 | 5 |
| 2-Methyl-1,3- | | | | 4 |
| propanediol | | | | |
| Butylene glycol | 3 | | | |
| Sodium EDTA | 0.18 | 0.18 | 0.18 | 0.18 |
| Aqueous sodium | pH 5.5 | pH 7.0 | pH 7.0 | pH 6.5 |
| hydroxide solution/citric | | | | |
| acid for pH adjustment | | | | |
| (quantum satis) | | | | |
| Water | to 100 | to 100 | to 100 | to 100 |
| Filling in 200 ml can | 94% emulsion | 96% emulsion | 95% emulsion | 94% emulsion |
| Propellants | 6% | 4% | 5% | 6% |
| Gas pressure rating | Propane/butane/ | Propane/butane/ | Propane/butane/ | Propane/butane/ |
| | isobutane | isobutane | isobutane | isobutane |
| | 2.7 bar | 3.0 bar | 3.0 bar | 3.5 bar |
| | G | H | I | J |
| Composition in % | m [%] | m [%] | m [%] | m [%] |
| Glyceryl stearate citrate | 2 | 2 | | |
| Sodium stearoyl | | | 0.2 | 0.4 |
| glutamate | | | | |
| Mineral oil | 1 | | | 1 |
| Caprylic/Capric | | | | |
| Triglyceride | 3 | 6 | 3 | 3 |
| Octyldodecanol | 4 | | 3 | 4 |
| Isopropyl Palmitate | | 3 | 3 | |
| Shea Butter | 1 | | 0.5 | 1 |
| Cocoa butter | | | 0.5 | |
| Coenzyme Q10 | 0.1 | | | 0.1 |
| Carbomer | 0.1 | 0.2 | 0.2 | 0.1 |
| Behenyl alcohol | | 2 | 2 | 2 |
| Ethanol | 5 | 2 | 3 | 1 |
| Glycerol | 5 | 7 | 9 | 10 |
| Phenoxyethanol | | 0.6 | | 0.5 |
| 1,2-Hexanediol | 1 | | | |
| 1,2-Pentanediol | | 1 | | |
| 1,2-Octanediol | | | 1.5 | |
| Perfume | 0.3 | | 0.3 | 0.35 |
| Sodium EDTA | 0.18 | 0.18 | 0.18 | 0.18 |
| Aqueous sodium | pH 5.5 | pH 7.0 | pH 7.0 | pH 6.5 |
| hydroxide solution for | | | | |
| pH adjustment (quantum | | | | |
| satis) | | | | |
| Water | to 100 | to 100 | to 100 | to 100 |
| Filling in 200 ml can | 94% emulsion | 95% emulsion | 94% emulsion | 95% emulsion |
| Propellants | 6% | 5% | 6% | 5% |
| Gas pressure rating | Propane/butane/ | Propane/butane/ | Propane/butane/ | Propane/butane/ |
| | isobutane | isobutane | isobutane | isobutane |
| | 3.0 bar | 3.0 bar | 3.5 bar | 2.7 bar |

1.-13. (canceled)

14. A cosmetic foam, wherein the foam comprises
(i) an O/W emulsion comprising (a) glycerol and (b) one or more mono- and/or polyhydric alcohols having a carbon chain length of less than 12 carbon atoms; and
(ii) a gas or gas mixture of propane, n-butane and/or isobutane which foams the O/W emulsion;
and wherein (i) is free of polyethylene glycol derivatives (PEG derivatives).

15. The foam of claim 14, wherein (i) comprises from 3% to 20% by weight of glycerol, based on a total weight of (i).

16. The foam of claim 14, wherein (i) comprises a total concentration of (b) of from 0.5% to 10% by weight, based on a total weight of (i).

17. The foam of claim 15, wherein (i) comprises a total concentration of (b) of from 0.5% to 10% by weight, based on the total weight of (i).

18. The foam of claim 14, wherein (b) comprises one or more of ethanol, phenoxyethanol, 1,2-pentanediol, 1,2-hexanediol, 1,2-octanediol, 2-methyl-1,3-propanediol, 1,2-decanediol, ethylhexylglycerol, benzyl alcohol, phenethyl alcohol.

19. The foam of claim 14, wherein (b) comprises ethanol.

20. The foam of claim 14, wherein (b) comprises phenoxyethanol.

21. The foam of claim 14, wherein (b) comprises ethylhexylglycerol.

22. The foam of claim 14, wherein (b) comprises 2-methyl-1,3-propanediol.

23. The foam of claim 14, wherein (b) comprises at least one of 1,2-pentanediol, 1,2-hexanediol, 1,2-octanediol.

24. The foam of claim 14, wherein (i) is free of soaps and surfactants having an HLB value of greater than 12.

25. The foam of claim 14, wherein the foam comprises from 90% to 96% by weight of (i) and from 4% to 10% by weight of (ii).

26. The foam of claim 14, wherein (i) is free of parabens, isothiazolinones, 3-iodopropargyl N-butylcarbamate (IPBC), and halogen-containing compounds.

27. The foam of claim 14, wherein a lipid phase of (i) comprises at least one of behenyl alcohol, almond oil, cocoa butter, shea butter.

28. The foam of claim 14, wherein (i) comprises from 7% to 25% by weight of lipid phase, based on a total weight of (i).

29. The foam of claim 14, wherein (i) comprises one or more emulsifiers selected from polyglycerol esters, cetearyl glycoside, sodium stearoyl glutamate, glyceryl stearate citrate, cetyl phosphate, stearic acid salts, a combination of sodium cetearyl sulfate and glyceryl stearate SE.

30. The foam of claim 14, wherein (i) comprises a total of from 0.1% to 8% by weight of one or more emulsifiers, based on a total weight of (i).

31. The foam of claim 14, wherein the foam is present in an aerosol can with release valve.

32. An aerosol can with release valve, wherein the can comprises the foam of claim 14.

33. A method of applying the foam of claim 31 to skin, wherein the method comprises shaking the can and subsequently withdrawing the foam via the release valve and applying it to the skin.
